SEQUENCE LISTING (6 sequences)

SEQ ID NO: 1 - RNA, 12 nt, synthetic construct
  source 1..12: mol_type = other RNA
tttttttctcgt

SEQ ID NO: 2 - RNA, 19 nt, synthetic construct
  source 1..19: mol_type = other RNA
atggtttgtttgtcttcgt

SEQ ID NO: 3 - RNA, 19 nt, synthetic construct
  source 1..19: mol_type = other RNA
atattctcccttgtttggt

SEQ ID NO: 4 - RNA, 12 nt, synthetic construct
  source 1..12: mol_type = other RNA
aataatgatcgg

SEQ ID NO: 5 - RNA, 15 nt, synthetic construct
  source 1..15: mol_type = other RNA
ttattttgttcgcgt

SEQ ID NO: 6 - RNA, 19 nt, synthetic construct
  source 1..19: mol_type = other RNA
tttttttctcgtttttacg

(19) United States
(12) Patent Application Publication
(10) Pub. No.: US 2024/0415923 A1
(43) Pub. Date: Dec. 19, 2024

(54) RNA-Dependent RNA Polymerase Inhibitory Compounds, Uses in Managing Viral Infections, and Pharmaceutical Compositions

(71) Applicant: Emory University, Atlanta, GA (US)

(72) Inventors: Bo Liang, Atlanta, GA (US); Dongdong Cao, Atlanta, GA (US); Eric Xu, Peachtree Corners, GA (US); Juan M. Calderon, Lawrenceville, GA (US); Seohyun Park, Suwanee, GA (US)

(21) Appl. No.: 18/742,659

(22) Filed: Jun. 13, 2024

Related U.S. Application Data
(60) Provisional application No. 63/472,637, filed on Jun. 13, 2023.
(60) Provisional application No. 63/621,814, filed on Jan. 17, 2024.

(51) Int. Cl. A61K 38/12; A61K 31/4155; A61K 31/549; A61K 39/42; A61P 31/14

(52) U.S. Cl. CPC A61K 38/12; A61K 31/4155; A61K 31/549; A61K 39/42; A61P 31/14

(57) ABSTRACT

Disclosed herein are methods of treating or preventing a viral infection comprising administering an effective amount an RNA-dependent RNA Polymerase (RdRP) inhibitory compound, derivative, or salt thereof as disclosed herein to a subject in need thereof. In certain embodiments, the viral infection is a positive or negative-sense RNA virus. In certain embodiments, the compound is administered in combination with an anti-viral agent or vaccine.

### CROSS-REFERENCE TO RELATED APPLICATIONS

This application claims the benefit of U.S. Provisional Application No. 63/472,637 filed Jun. 13, 2023 and U.S. Provisional Application No. 63/621,814 filed Jan. 17, 2024. The entirety of each of these applications is hereby incorporated by reference for all purposes.

### STATEMENT REGARDING FEDERALLY SPONSORED RESEARCH OR DEVELOPMENT

This invention was made with government support under GM130950 awarded by the National Institutes of Health. The government has certain rights in this invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 23176US.xml. The XML file is 6 KB, was created on Jun. 13, 2024, and is being submitted electronically via the USPTO patent electronic filing system.

### BACKGROUND

Respiratory syncytial virus (RSV) is a common cold virus belonging to the family of paramyxovirus. RSV is a major cause of hospital visits for premature babies and newborns. RSV infections also pose a threat for the elderly and immune compromised. Children are at risk for severe RSV lower respiratory tract infection (LRTI). Major risk factors for hospitalization due to RSV infections are premature birth, age younger than 6 weeks, chronic lung disease (CLD), congenital heart disease, and compromised immunity.

Vaccines are typically killed (inactivated) or weakened (attenuated) versions of a live viral strain. Attenuated RSV vaccine candidates face significant safety hurdles, and the development of pediatric RSV live-attenuated vaccine (LAV) strains that are sufficiently attenuated and immunogenic have been elusive. See Collins et al. Progress in understanding and controlling respiratory syncytial virus: still crazy after all these years. Virus Res, 2011,162, 80-99.

Palivizumab (Synagis®) is a humanized monoclonal antibody targeting the fusion (F) protein of RSV. Palivizumab is indicated for use in high-risk children: preterm infants 35 weeks or less gestational age (GA), children with CLD of prematurity, and children with hemodynamically significant congenital heart disease (CHD). Palivizumab has limited efficacy and sometimes causes allergic reactions. In the U.S., palivizumab is not recommended for healthy preterm infants. As there is no approved RSV prophylaxis for the broader population of healthy infants and no treatment for RSV, the current management for these patients when they acquire serious RSV illness is supportive care. Thus, there remains a need to identify improved therapies.
Bonavia et al. report broad-spectrum antiviral compounds and assessments for efficacy against respiratory syncytial virus (RSV). Proc Natl Acad Sci USA, 2011, 108, 6739-6744.
Kim et al. report antiviral activity of micafungin against enterovirus. Virol J, 2016, 13, 99.
Lu et al. report antiviral effects of combination treatments against Zika virus infection. Antiviral Res, 2021, 195, 105188.
Chen et al. report micafungin inhibits dengue virus infection through the disruption of virus binding, entry, and stability. Pharmaceuticals, 2021, 14, 338.
Mir et al. report successful treatment of respiratory syncytial virus infection in an immunocompromised patient with ribavirin. Curcus, 2021, 13 (8): e16930.
Chandrasekar and Sobel report micafungin is an inhibitor of 1,3-β-D-glucan synthase. Clinical Infectious Diseases, 2006, 42 (8): 1171-1178.
Garzon et al. report use of thrombopoietin receptor agonists in childhood immune thrombocytopenia. Front Pediatr, 2015, 3, 70.
Oblak et al. report the prophylactic evaluation of verubecestat on disease- and symptom-modifying effects in mice. Alzheimer's Dement, 2022, 8: e12317.
Sudo et al. report YM-53403 is an anti-respiratory syncytial virus agent. Antiviral Res, 2005, 65 (2): 125-31.
Troke et al. report resistance to the nonnucleoside NS5B inhibitor filibuvir in hepatitis C virus-infected patients. Antimicrob Agents Chemother, 2012, 56 (3): 1331-1341.
Oey et al. report lumicitabine, an orally administered nucleoside analog, in infants hospitalized with respiratory syncytial virus (RSV) infection. PLOS ONE, 2023, 18 (7): c0288271.

References cited herein are not an admission of prior art.

### SUMMARY

Disclosed herein are methods of treating or preventing a viral infection comprising administering an effective amount an RNA-dependent RNA Polymerase (RdRP) inhibitory compound, derivative, or salt thereof as disclosed herein to a subject in need thereof.

In certain embodiments, the viral infection is a positive-sense or negative-sense RNA virus, non-segmented negative-sense (NNS) RNA virus such as respiratory syncytial virus (RSV), vesicular stomatitis virus (VSV), Nipah virus (NiV), variegated squirrel bornavirus (VSBV), coronavirus, severe acute respiratory syndrome coronavirus 2 (SARS-COV2), rabies virus (RABV), or ebola virus (EBOV). In certain embodiments, the compound is administered in combination with an anti-viral agent or vaccine.

In certain embodiments, the viral infection is respiratory syncytial virus (RSV) infection, and the inhibitory compound is selected from: micafungin, PC786, AZ-27, totrombopag, verubecestat, trovafloxacin, ribavirin, ALS-8112, azlocillin, trospium, amiodarone, perflubron, mephenesin, methadone, YM-53403, filibuvir, remdesivir-TP, ALS-8112, ALS-8176 (lumicitabine), or combinations thereof.

In certain embodiments, this disclosure relates to pharmaceutical compositions comprising compounds disclosed herein. In certain embodiments, this disclosure relates to pharmaceutical compositions comprising an RNA-dependent RNA Polymerase (RdRP) inhibitory compound, derivative, or salt thereof as reported herein and optionally another pharmaceutical agent, e.g., an anti-viral agent or anti-RSV agent.

### BRIEF DESCRIPTION OF THE SEVERAL VIEWS OF THE DRAWINGS

FIG. 1 shows a table of candidate molecules as select inhibitors identified using computer calculations (AutoDock™ 4.2) that predicted inhibition constants (nM).

FIG. 2 shows quantitative data from an in vitro RNA synthesis assay using RSV polymerase. Testing was performed with different candidate molecules using 12-nt RNA (TrC12) 5′ UUUUUUUCUCGU (SEQ ID NO: 1) as the template. Verubecestat, Micafungin, Filibuvir, Ribavirin, ALS-8112, and ALS-8176 at a concentration of 1 mM were tested with RSV polymerase using TrC12 as a template in the presence of NTPs (GTP, CTP, and UTP each at 1.25 mM and GTP at 50 μM with 5 μCi of [α-32P] ATP). The molecules were dissolved in 10% dimethyl sulfoxide (DMSO), and 10% DMSO was used as a negative control. Total polymerase activities were quantified and plotted.

FIG. 3A shows data from in vitro RNA synthesis assay testing the RNA synthesis activities of RSV polymerase in the presence of different small molecules. The de novo RNA synthesis assay tests different small molecules using TrC12 as a template. The reaction mixtures were incubated in the presence of NTPs (GTP, CTP, and UTP, each at 1.25 mM and ATP at 50 μM with 5 μCi of [a-32P] ATP).

FIG. 3B shows data from the primer-based RNA synthesis assay that tests different small molecules using TrC12 as a template and Tr4 as a primer. The reaction mixtures were incubated in the presence of 5 μCi of [a-32P] ATP). The RNA template TrC12 which is derived from the promoter of RSV antigenome. Tr4 (5′-ACGA) is complementary to the first 4 nts of TrC12. The small molecules, including Totrombopag, Verubecestat, Micafungin, YM-53403, PC786, Filibuvir, Ribavirin, Remdesivir-TP, EIDD-1931-TP, ALS-8112, and ALS-8176, are all 1 mM in the final reaction mixture.

FIG. 4A shows data from the in vitro RNA synthesis assay testing compounds with viral RNA polymerases from the VSV virus. The de novo RNA synthesis assay tests compound with VSV polymerase (L-P1-106) using VSV Le19 5′-AUGGUUUGUUUGUCUUCGU (SEQ ID NO: 2) as a template. The reaction mixtures in panel were incubated in the presence of NTPs (GTP, CTP, and UTP each at 1.25 mM and ATP at 50 μM with 5 μCi of [a-32P] ATP). Micafungin, Verubecestat, and Totrombopag, are 1 mM in the final reaction mixtures.

FIG. 4B shows data from the in vitro RNA synthesis assay testing compounds with NiV polymerase (L-P) using NiV Le19 5′AUAUUCUCCCUUGUUUGGU (SEQ ID NO: 3) as a template. The reaction mixtures were incubated in the presence of NTPs (GTP, CTP, and UTP, each at 1.25 mM and ATP at 50 μM with 5 μCi of [a-32P] ATP). Micafungin, Verubecestat, and Totrombopag, are all 1 mM in the final reaction mixtures. The reaction mixtures in panel were incubated in the presence of NTPs (GTP, CTP, and UTP each at 1.25 mM and ATP at 50 μM with 5 μCi of [a-32P] GTP). Micafungin, Verubecestat, and Totrombopag are 1 mM in the final reaction mixtures.

FIG. 4C shows data from the in vitro RNA synthesis assay testing compounds with VSBV polymerase (L-P) using VSBV Le12 5′-AAUAAUGAUCGG (SEQ ID NO: 4) as a template. Micafungin, Verubecestat, and Totrombopag, are all 1 mM in the final reaction mixtures. The reaction mixtures in panel were incubated in the presence of NTPs (GTP, CTP, and UTP each at 1.25 mM and GTP at 50 μM with 5 μCi of [a-32P] ATP). Micafungin, Verubecestat, and Totrombopag, are 1 mM in the final reaction mixtures.

FIG. 4D shows data from the in vitro RNA synthesis assay testing compounds with SARS-COV2 polymerase (Nsp7-Nsp8-Nsp12) using T1 5′-UUAUUUUGUUCGCGU (SEQ ID NO: 5) as a template. The reaction mixtures in panel were incubated in the presence of NTPs (GTP, CTP, and UTP each at 1.25 mM and GTP at 50 μM with 5 μCi of [a-32P] ATP). Micafungin, Verubecestat, and Totrombopag, are 1 mM in the final reaction mixtures.

FIG. 5A data on RSV polymerase activities from in vitro RNA synthesis assay evaluating the time course of varying Micafungin concentrations (time course of 0, 25, and 50 μM Micafungin using TrC12 as a template). The reaction mixtures were incubated in the presence of NTPs (GTP, CTP, and UTP, each at 1.25 mM and ATP at 50 μM with 5 μCi of [a-32P] ATP).

FIG. 5B shows data comparing RSV polymerase activities of Micafungin and PC786 by in vitro RNA synthesis assays. The top figure shows results of the primer-based RNA synthesis assay tests with varying Micafungin concentrations using 5′-FAM-TrC12-4U-Tr3 (5′-FAM-UUUUUUUCUCGUUUUUACG) (SEQ ID NO: 6) as a substrate. The reaction mixtures were incubated with varying concentrations of Micafungin (2% DMSO in the final mixture) in the presence of ATP. The bottom figure shows results of the primer-based RNA synthesis assay tests with varying PC786 concentrations using 5′-FAM-TrC12-4U-Tr3 as a substrate. The reaction mixtures were incubated with varying concentrations of PC786 (4% DMSO in the final mixture) in the presence of ATP.

### DETAILED DISCUSSION

An “embodiment” is an example of this disclosure and not necessarily limited to such example. It is also to be understood that the terminology used herein is for describing particular embodiments only, and is not intended to be limiting, since the scope of the present disclosure will be limited only by the appended claims or as amended during prosecution. No language in the specification should be construed as indicating any non-claimed element as essential to the practice of the invention. The use of any and all examples, or exemplary language (e.g., “such as”) provided herein, is intended merely to better illuminate the invention and does not pose a limitation on the scope of the invention unless otherwise claimed.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The dates of publications provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible. All methods described herein can be performed in any suitable order unless otherwise indicated herein or otherwise clearly contradicted by context.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Use of the term “about” is intended to describe values either above or below the stated value in a range of approximately plus or minus 10%; in other embodiments the values may range in value either above or below the stated value in a range of approximately plus or minus 5%. The preceding ranges are intended to be made clear by context, and no further limitation is implied.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to methods and compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

As used herein, a “subject” refers to any animal, preferably a human patient, livestock, or domestic pet. In certain embodiments, the subject is a human subject less than 2, 12, or 16 years old or 2, 12, or 15 years old or older. In certain embodiments, the subject is a human subject 55 or 65 years old or older. In certain embodiments, the subject is a human subject greater than 55, 60, 65, or 70 years of age. In certain embodiments, the subject is an infant, e.g., from one month to two years of age. In certain embodiments, the subject is a human subject such as a child, e.g., from two to twelve years of age. In certain embodiments, the subject is a human subject such as an adolescent, e.g., from twelve to sixteen years of age. In certain embodiments, the subject is a human subject sixteen years of age or older.

As used herein, an “infant” subject is a human subject that is one year (12 months) of age or younger. As used herein, a “pediatric” subject is a human child subject that is older than one year of age. In some embodiments, a pediatric subject is a human subject that is older than one year (12 months) of age and up to or including 24 months of age.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

As used herein, the term “derivative” refers to a structurally similar compound that retains sufficient functional attributes of the identified analogue. The derivative may be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, but not limited to, replacing an oxygen atom with a sulfur atom, or replacing an amino group with a hydroxyl group. The derivative may be a prodrug, alkyl ester, alkanoyl ester, comprise a lipid, polyethylene glycol, saccharide, or polysaccharide.

### Methods of Use

This disclosure relates to methods of treating or preventing a viral infection comprising administering an effective amount an RNA-dependent RNA Polymerase (RdRP) inhibitory compound, derivative, or salt thereof to a subject in need thereof, wherein the compound is selected from:
5-(2-(20-(3-amino-1-hydroxy-3-oxopropyl)-2,11,12,15-tetrahydroxy-6-(1-hydroxyethyl)-16-methyl-5,8,14,19,22,25-hexaoxo-9-(4-(5-(4-(pentyloxy)phenyl) isoxazol-3-yl)benzamido)tetracosahydro-1H-dipyrrolo[2,1-c: 2′,1′-1][1,4,7,10,13,16]hexaazacyclohenicosin-23-yl)-1,2-dihydroxyethyl)-2-hydroxyphenyl hydrogen sulfate (Micafungin);
N-(2-fluoro-6-methylphenyl)-6-(4-(5-methyl-2-(7-oxa-2-azaspiro[3.5]nonan-2-yl) nicotinamido)benzoyl)-5,6-dihydro-4H-benzo[b]thieno[2,3-d]azepine-2-carboxamide (PC786);
6-(4-(2-(2-oxa-7-azaspiro[3.5]nonan-7-yl) nicotinamido)benzoyl)-N-cyclopropyl-5,6-dihydro-4H-benzo[b]thieno[2,3-d]azepine-2-carboxamide (AZ-27);
2-(3,4-dimethylphenyl)-4-((2-hydroxy-3′-(1H-tetrazol-5-yl)-[1,1′-biphenyl]-3-yl)diazenyl)-5-methyl-1,2-dihydro-3H-pyrazol-3-one (Totrombopag);
N-(3-(3-amino-2,5-dimethyl-1,1-dioxido-5,6-dihydro-2H-1,2,4-thiadiazin-5-yl)-4-fluorophenyl)-5-fluoropicolinamide (Verubecestat);
7-((6-amino-3-azabicyclo[3.1.0]hexan-3-yl)-1-(2,4-difluorophenyl)-6-fluoro-4-oxo-1,4-dihydro-1,8-naphthyridine-3-carboxylic acid (Trovafloxacin);
1-(3,4-dihydroxy-5-(hydroxymethyl)tetrahydrofuran-2-yl)-1H-1,2,4-triazole-3-carboxamide (Ribavirin);
4-amino-1-(5-(chloromethyl)-3-fluoro-4-hydroxy-5-(hydroxymethyl)tetrahydrofuran-2-yl)pyrimidin-2 (1H)-one (ALS-8112);
3,3-dimethyl-7-oxo-6-(2-(2-oxoimidazolidine-1-carboxamido)-2-phenylacetamido)-4-thia-1-azabicyclo[3.2.0]heptane-2-carboxylic acid (Azlocillin);
3-(2-hydroxy-2,2-diphenylacetoxy)spiro[bicyclo[3.2.1]octane-8,1′-pyrrolidin]-8-ium (Trospium);
(2-butylbenzofuran-3-yl) (4-(2-(diethylamino) ethoxy)-3,5-diiodophenyl) methanone (Amiodarone);
1-bromo-1,1,2,2,3,3,4,4,5,5,6,6,7,7,8,8,8-heptadecafluorooctane (Perflubron);
3-(o-tolyloxy) propane-1,2-diol (Mephenesin);
6-(dimethylamino)-4,4-diphenylheptan-3-one (Methadone);
6-(4-([1,1′-biphenyl]-2-carboxamido)benzoyl)-N-cyclopropyl-5,6-dihydro-4H-benzo[b]thieno[2,3-d]azepine-2-carboxamide (YM-53403);
6-cyclopentyl-6-(2-(2,6-diethylpyridin-4-yl)ethyl)-3-((5,7-dimethyl-[1,2,4]triazolo[1,5-a]pyrimidin-2-yl)methyl)-4-hydroxy-5,6-dihydro-2H-pyran-2-one (Filibuvir);
5-(4-aminopyrrolo[2,1-f][1,2,4]triazin-7-yl)-5-cyano-3,4-dihydroxytetrahydrofuran-2-yl)methyl tetrahydrogen triphosphate (Remdesivir triphosphate);
1-(3,4-dihydroxy-5-(hydroxymethyl)tetrahydrofuran-2-yl)-4-(hydroxyimino)-3,4-dihydropyrimidin-2 (1H)-one triphosphate (EIDD-1931-TP);
5-(4-amino-2-oxopyrimidin-1 (2H)-yl)-2-(chloromethyl)-4-fluoro-2-((isobutyryloxy)methyl)tetrahydrofuran-3-yl isobutyrate (Lumicitabine); or derivatives thereof.

In certain embodiments, compound disclosed herein is administered in combination with another anti-viral agent such as, abacavir, acyclovir, adefovir, amantadine, arbidol, baloxavir, boceprevir, daclatasvir, docosanol, edoxudine, enfuvirtide, famciclovir, foscarnet, ganciclovir, ibacitabine, idoxuridine, imiquimod, imunovir, lamivudine, letermovir, marboxil, methisazone, moroxydine, nexavir, oseltamivir, peramivir, penciclovir, pleconaril, ribavirin, rimantadine, simeprevir, sofosbuvir, taribavirin, telbivudine, tenofovir, trifluridine, tromantadine, umifenovir, valaciclovir, vidarabine, zanamivir, zidovudine, or combinations thereof.

In certain embodiments, the viral infection is respiratory syncytial virus (RSV) infection. In certain embodiments, the compound is administered in combination with an anti-RSV agent or an RSV-vaccine. In certain embodiments, the anti-RSV agent is ribavirin, lumicitabine, or YM-53403. In certain embodiments, the anti-RSV agent is a recombinant RSV antibody. In certain embodiments, the recombinant RSV antibody is palivizumab or nirsevimab (e.g., nirsevimab-alip—nirsevimab and excipients L-histidine, L-histidine salts, and L-arginine salts, sucrose, polysorbate, for water-based injection).

In certain embodiments, the subject is pregnant woman, a premature baby, newborn, infant, child, adult, elderly, and/or immune compromised. In certain embodiments, the subject is a human such as an infant or pediatric patient that experienced a premature birth. In certain embodiments, the subject is a preterm infant 35 or 30 weeks or less gestational age (GA), premature baby with long-term breathing problems, bronchopulmonary dysplasia (BPD), or child with hemodynamically significant congenital heart disease (CHD). In certain embodiments, the subject is diagnosed with RSV and chronic lung disease (CLD), congenital heart disease, compromised immunity, at an age younger than 6 weeks, at an age older than 50 or 60 years, or is pregnant.

In certain embodiments, the subject is immune compromised due to the need to maintain an immune suppressive drug(s) therapy, e.g., the subject is or is diagnoses with DiGeorge syndrome, Wiskott-Aldrich syndrome, Bruton's agammaglobulinemia, the subject is receiving (being administered) chemotherapy or radiation due to being diagnosed with cancer, the subject is receiving corticosteroids due to a diagnosis of rheumatoid arthritis, lupus, vasculitis, or the subject is a solid organ recipient that is taking (e.g., self-administered or by infusion) immune suppressive drugs to prevent rejection of a transplanted organ.

In certain embodiments, the method comprises preventing very severe RSV or other infection in an infant or pediatric subject or immune compromised patient or elderly patient in need thereof. In certain embodiments, the subject is a human patient, e.g., newborn less than 1 years old. In certain embodiments, the human subject experienced, is at risk of, or diagnosed with premature birth, chronic lung disease (CLD), congenital heart disease, compromised immunity, or at an age younger than 6 weeks old. In certain embodiments, the subject is a human patient, e.g., newborn less than 1 years old. In certain embodiments, the subject is more than 1 years old. In some embodiments, the compound is administered at birth.

In some embodiments, the prophylactic methods reported herein include administering a compound as provided herein to prevent viral lower respiratory tract infection such as an RSV-associated lower respiratory tract infection (LRTI). In some embodiments, the prophylactic methods herein prevent viral-associated or RSV-associated hospitalization. In certain embodiments, the compounds disclosed herein are infants entering their first RSV season and children at a high risk of developing RSV infections e.g., children with chronic liver disease (CLD) or chronic heart disease CHD) entering their first or second RSV season.

In certain embodiments, this disclosure relates to methods of diagnosing a subject with a viral infection and treating the subject with a compound or salt as disclosed herein. The infection (e.g., a positive viral test) can be determined by diagnostic methods known in the art. Sec, e.g., Midgley et. al., Determining the Seasonality of Respiratory Syncytial Virus in the United States: The Impact of Increased Molecular Testing. J Infect Dis. 2017. In some embodiments, a test may be performed on an upper respiratory sample. In some embodiments, a test may be performed on a lower respiratory sample. In some embodiments, the viral infection is determined by a polymerase chain reaction (PCR)-based method. In some embodiments, the viral infection is determined by an antigen-based method. In some embodiments, infection is determined by virus isolation by culture. In some embodiments, infection is determined by serological testing.

In some embodiments, a “severe infection” is characterized by at least one of the following: increased respiratory rate (greater than 60 breaths/min for less than 2-month-old; greater than 50 breaths/min for 2-month old to 6-month old, greater than 40 breaths/min for 6-month old to 24-month old); hypoxemia in room air (O2 less than 95% at less than 1800 m; O2 less than 92% at greater than 1800 m); new-onset apnea; retractions; grunting; nasal flaring; acute hypoxic or ventilatory failure; dehydration due to respiratory distress requiring intravenous hydration; intercostal, subcostal, or supraventricular retractions. In some embodiments, severe infection is characterized by hospitalization for a medically attended patient.

As used herein, a “very severe infection” is characterized by hospitalization for medically attended patient and requiring supplemental oxygen and/or intravenous fluids. In some embodiments, a “very severe infection” is characterized by oxygen saturation (SaCh) less than 90%.

In certain embodiments, the drug/compound, derivative, or salt thereof as reported herein or pharmaceutical composition is administered by inhalation, orally, systemically, or locally, i.e., parenteral, subcutaneous, intravenous, intramuscular, intradermal, intranasal, aerosolized, or any other path of administration.

In certain embodiment this disclosure relates to methods of treating positive-sense virus or negative-sense RNA virus comprising administering an effective amount of a compound disclosed herein to a subject in need thereof. In certain embodiments, the compound is micafungin and/or totrombopag.

In certain embodiment, this disclosure relates to methods of treating or preventing a respiratory syncytial virus (RSV) infection or other viral infection comprising administering an effective amount of micafungin and/or totrombopag or other compound disclosed herein to a subject in need thereof. In certain embodiments, micafungin and/or totrombopag are administered in combination with an anti-viral agent, anti-RSV agent or vaccine. In certain embodiments, the anti-RSV agent is ribavirin, lumicitabine, YM-53403, or a recombinant RSV antibody.

In certain embodiment, this disclosure relates to methods of treating or preventing a severe acute respiratory syndrome coronavirus 2 (SARS-COV2/COVID-19) or other coronavirus infection comprising administering an effective amount of micafungin or other compound disclosed herein to a subject in need thereof. In certain embodiments, a subject in need thereof is diagnosed with SARS-COV-2, SARS-COV-1, MERS—COV, HCOV-229E, HCoV-OC43, HCoV-NL63, or HCoV-HKU1, an endemic human coronavirus, epidemic coronavirus, or pandemic coronavirus.

In certain embodiments, the subject is diagnosed with a viral infection that poses a risk of developing an acute respiratory syndrome or chronic acute respiratory syndrome such as subject diagnosed with a high-risk coronavirus infection, e.g., SARS-COV-1 or SARS-COV-2 infection.

In certain embodiments, the subject is diagnosed with fatigue, shortness of breath, anxiety, depression, brain fog, joint pain, and/or chest pain, and optionally diabetes, stroke, heart rhythm abnormality, and/or blood clot in the lungs.

In certain embodiments, a compound disclosed herein can be administered in combination with other anti-viral or anti-coronaviral agents such as remdesivir, chloroquine, hydroxychloroquine, azithromycin, ivermectin, lopinavir, ritonavir, nitazoxanide, molnupiravir, or combinations thereof.

In certain embodiments, a compound disclosed herein can be administered in combination with other anti-viral agents such as nirmatrelvir and/or ritonavir. In certain embodiments, treatment commences within 5 days or 7 days of when symptoms start.

In certain embodiments, a compound disclosed herein can be administered in combination with remdesivir. In certain embodiments, treatment commences within 5 days or 7 days of when symptoms start. In certain embodiments, a compound disclosed herein can be administered in combination with molnupiravir. In certain embodiments, treatment commences within 5 days or 7 days of when symptoms start.

In certain embodiments, a compound disclosed herein can be administered in combination with convalescent plasma. In certain embodiments, the subject is prone to an overactive immune response, or the subject is immunocompromised or are receiving immunosuppressive treatment.

In certain embodiments, it is contemplated that the subject may have a viral and a bacterial infection, e.g., pneumonia. In certain embodiments, this disclosure contemplates administering a compound reported herein or salt thereof, in combination with an anti-bacterial agent.

In certain embodiments, micafungin and/or totrombopag are administered in combination with an anti-SARS-COV2 agent or vaccine. In certain embodiments, the anti-SARS-COV2 agent is remdesivir, chloroquine, hydroxychloroquine, azithromycin, ivermectin, lopinavir, ritonavir, nitazoxanide, molnupiravir or a recombinant SARS-COV2 antibody.

In certain embodiments, this disclosure contemplates methods of treating or preventing a Nipah virus (NiV) infection comprising administering an effective amount of micafungin or other compound disclosed herein and optionally another antiviral agent to a subject in need thereof.

In certain embodiments, this disclosure contemplates methods of treating or preventing an Ebola virus (EBOV) infection comprising administering an effective amount of micafungin or other compound disclosed herein and optionally another antiviral agent to a subject in need thereof. In certain embodiments, treatment is in combination with another anti-Ebola therapeutic such as an anti-Ebola antibody or combination of antibodies, e.g., Inmazeb™ (atoltivimab, maftivimab, and odesivimab-ebgn) or Ebanga™ (ansuvimab-zykl).

In certain embodiments, this disclosure contemplates methods of treating or preventing a variegated squirrel bornavirus (VSBV) infection comprising administering an effective amount of micafungin or other compound disclosed herein and optionally another antiviral agent to a subject in need thereof.

In certain embodiments, this disclosure contemplates methods of treating or preventing a rabies virus (RABV) infection comprising administering an effective amount of micafungin or other compound disclosed herein and optionally another antiviral agent to a subject in need thereof. In certain embodiments, the compound is administered in combination with an antiviral agent, human rabies immune globulin (HRIG), or vaccine.

In certain embodiments, the subject is more than 55, 65, or 75 years old and/or diagnosed with a severe acute infection requiring intensive care, pre-existing respiratory illness, obesity, diabetes, high blood pressure, chronic cardiovascular disease, chronic kidney disease, organ transplant, or cancer.

Although embodiments of this disclosure contemplate treatment of certain virus, e.g., RSV and coronavirus infections, management of other viral infections are contemplated such as influenza virus, rhinovirus, hepatitis A, hepatitis B, hepatitis C, human papillomaviruses, herpes virus, Epstein-Barr virus, herpes simplex virus, varicella-zoster virus, shingles virus, mumps virus, measles virus, West Nile virus, poliovirus, non-poliovirus enterovirus, and parainfluenza virus.

### Pharmaceutical Compositions and Kits

In certain embodiments, this disclosure relates to pharmaceutical compositions for use in treating a viral infection comprising a compound, derivative, or salt thereof as reported herein. The pharmaceutical compositions provided herein may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

In certain embodiments, this disclosure relates to a pharmaceutical composition having a compound, derivative, or salt thereof as disclosed herein and pharmaceutically acceptable excipient selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a sterilized pH buffered aqueous salt solution or a saline phosphate buffer between a pH of 6 to 8, optionally comprising a saccharide or polysaccharide. In certain embodiments, the formulations are isotonic in relation to human blood. Isotonic solutions possess the same or similar concentration of salts and water for maintaining osmotic balance with blood plasma so that they can be intravenously infused into a subject without substantially changing the osmotic pressure of the blood plasma.

In certain embodiments, this disclosure relates to a pharmaceutical composition having a compound, derivative, or salt thereof as disclosed herein and a pharmaceutically acceptable carriers such as alumina, aluminum stearate, lecithin, serum proteins, albumin, such as human serum albumin, buffer substances such as phosphates, glycine, sorbic acid, citric acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, hydrophilic polymers such as polyvinyl pyrrolidone, cellulose based substances, polyethylene glycol, sodium carboxymethylcellulose, polyacrylates, waxes, gelatin, polyethylene polyoxypropylene block polymers, polyethylene glycol and antioxidants including ascorbic acid and methionine; preservatives; low molecular weight (less than about 10 residues) polypeptides; proteins; and amino acids such as glycine, glutamine, asparagine, histidine, arginine, or lysine. In certain embodiments, the excipient may be one or more selected from the list consisting of NaCl, trehalose, sucrose, mannitol, or glycine.

In certain embodiments, this disclosure relates to pharmaceutical compositions comprising a compound, derivative, or salt thereof as reported herein and another pharmaceutical agent, e.g., an anti-viral or anti-RSV agent. In certain embodiments, the anti-RSV agent is ribavirin or a recombinant RSV antibody. In certain embodiments, the recombinant RSV antibody is palivizumab or nirsevimab-alip.

In certain embodiments, the compound, derivative, or salt thereof is formulated in a lipid particle. In certain embodiments, the lipid particle is made up of an ionizable lipid, a phospholipid, a sterol, e.g., cholesterol, and a polyethylene glycosylated lipid.

In certain embodiments, the pharmaceutical composition optionally includes an adjuvant, i.e., a non-specific stimulator of the immune response, or administered in combination with an adjuvant. Contemplated adjuvants suitable for depot and delivery are cationic or polycationic compounds, liposomes, chitosan, alum solution, aluminium hydroxide, aluminium salts, aluminium phosphate gel, aluminium hydroxide gel (alum), polyphosphazene, squalene, squalene water emulsion, CpG oligonucleotides (nucleic acids with unmethylated CpG motifs), 1-alpha 25-dihydroxyvitamin D3, calcium phosphate gel, dimethyl dioctadecyl ammonium bromide, dehydroepiandrosterone, dimyristoylphosphatidylcholine, myristoyl phosphatidylglycerol, deoxycholic acid sodium salt, imiquimod, interferon gamma, interleukin-l beta, interleukin-2, interleukin-7, interleukin-12, 7-allyl-8-oxoguanosine, acetylmuramyl-alanyl-isoglutamine, N-acetyl muramyl-L-threonyl-D-isoglutamine, NAc-Mur-L-Ala-D-Gln-OCH3, QS-21, Quil-A (Quil-A saponin), sorbitan trioleate, 2,6, 10,15, 19,23-hexamethyltetracosan, stearyl tyrosine, lipid A, 4′-monophosphoryl lipid A, 3-O-desacyl-4′-monophosphoryl lipid A (MPLTM), liposomes containing lipid A, lipid A adsorbed on aluminium hydroxide.

In certain embodiments, this disclosure relates to kits containing a compound, derivative, or salt thereof as reported herein useful for the treatment, or prevention of an RSV infection. In certain embodiments, the kit comprises a container, a product label and a package insert. Suitable containers include, for example, bottles, vials, syringes, and boxes containing the same. In certain embodiments, the containers may be of a variety of materials, e.g., glass, plastic, or cardboard. In certain embodiments, the container holds the composition which is effective in treating the condition and may have a sterile access port (for example the container may be an intravenous solution bag or a vial having a stopper pierceable by a hypodermic injection needle). In certain embodiments, at least one active agent in the composition is a RdRP inhibitory compound, derivative, or salt thereof as disclosed herein. In certain embodiments, the product label on, or associated with, the container indicates that the composition is used for treating the condition of choice.

In certain embodiments, the kit may further comprise a second container comprising a pharmaceutically acceptable buffer, such as a phosphate buffer saline or a citrate buffered saline. It may further include other materials desirable for a user or from commercial standpoint, including other buffers, diluents, filters, needles, and syringes. In certain embodiments, a dosage unit form can be, e.g., in the format of a prefilled syringe, an ampoule, cartridge or a vial. Preferably, the container, vial, or syringe is composed of glass, plastic, or a polymeric material chosen from an olefin polymer or copolymer. The syringe, ampoule, cartridge, or vial can be manufactured of any suitable material, such as glass or plastic and may include rubber materials, such as rubber stoppers for vials and rubber plungers and rubber seals for syringes and cartridges.

In certain embodiments, this disclosure relates to kits or articles of manufacture, comprising a compound, derivative, or salt thereof as disclosed herein and instructions for use by, e.g., the patient or a healthcare professional. In certain embodiments, the kit may further comprise instructions for use and/or a clinical package leaflet. In certain embodiments, this disclosure also relates to packaging material, instructions for use, and/or clinical package leaflets, e.g., as required by regulatory aspects.

### In Silico Identification and In Vitro Validation of Repurposed Compounds Targeting the RSV Polymerase

The RNA-dependent RNA Polymerase (RdRP) of RSV is needed for replication and transcription. Based on the cryo-EM structure of the RSV polymerase, in silico computational analysis including molecular docking and the protein-ligand simulation of a database was performed and has resulted in repurposed compound candidates against the RSV polymerase, including micafungin, totrombopag, and verubecestat. Among the compounds, micafungin showed significant inhibition and binding affinity improvements over inhibitors such as ALS-8112 and ribavirin. The inhibition of RSV RdRP with micafungin was validated using an in vitro transcription assay. These findings indicate the potential use as a broad-spectrum antiviral agent targeting the non-segmented negative-sense (NNS) RNA viral polymerases, including those of rabies (RABV) and Ebola (EBOV).

The Respiratory Syncytial Virus (RSV) is the most prominent cause of infant hospitalization, bronchiolitis, and pneumonia, while also being a major cause of hospitalization for immunocompromised individuals and the elderly. RSV has a negative-sense RNA genome encapsidated by nucleoproteins. The virus is highly contagious through the respiratory route, leading down to the nasopharynx and upper respiratory tract.

The L protein, the catalytic core of the RNA-dependent RNA Polymerase (RdRP), is usually a primary target against viral infection. The RdRP begins replication via cis-acting sequences in the Le region adjacent to the 30 termini. In transcription, the polymerase starts at the 30 termini and stops at the gene end before restarting at gene start signals, synthesizing messenger RNA (mRNA) along the nucleocapsid. During replication, the polymerase ignores the gene junctions and synthesizes a full-length complementary antigenome RNA; the encapsidated antigenome then increases polymerase processivity to allow RNA synthesis.

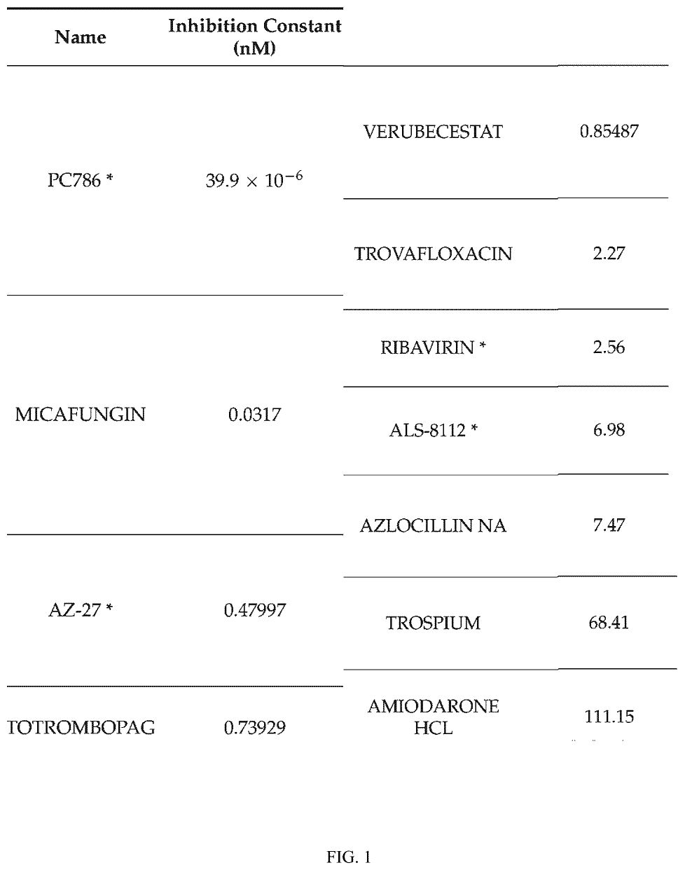

The ChEMBL database was used to select inhibitor candidates that had undergone clinical trials from Phases 1 to 4, ensuring minimal cytotoxicity. After reducing these to a more limited number of candidates by elimination of flexible ligands, binding affinities of the compounds were calculated and ranked providing top candidates (FIG. 1). Micafungin, totrombopag, and verubecestat emerged as promising compound candidates based on their binding affinities.

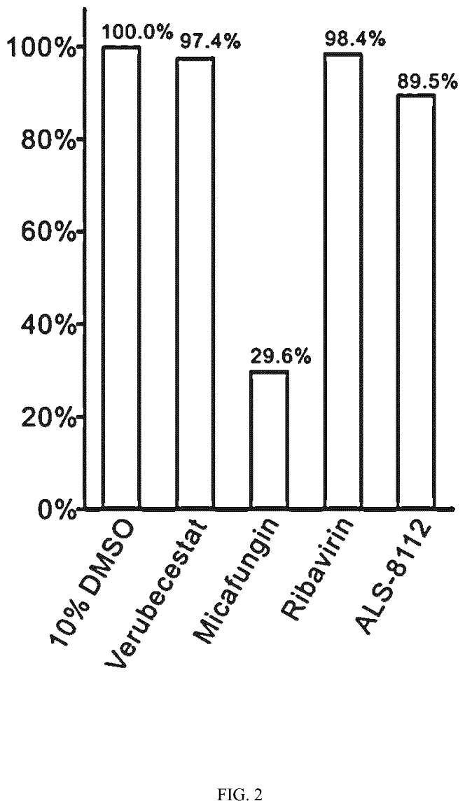

Using an in vitro assay, select compounds were validated (FIG. 2). Micafungin exhibited significant inhibitory activity. Verubecestat showed inhibition. These compounds are contemplated as broad-spectrum antivirals targeting the polymerases viruses such as rabies (RABV) and Ebola (EBOV).

### In Vitro RNA Synthesis Assay Screening Inhibitors

The 12-nt trailer complementary sequence (TrC12) at the 30 termini of the antigenome was used as a template in the RNA synthesis assay. RNA oligonucleotides were chemically synthesized. The reaction mixtures contained a 2 μM RNA template TrC12, the RSV L-P complexes (u300 ng), NTPs (GTP, CTP, and UTP each at 1.25 mM and ATP at 50 μM with 5 μCi of [32P] ATP), 1 mM small molecule in 10% DMSO (10% DMSO solution was used as a negative control), and a reaction buffer (50 mM Tris-HCl pH 7.4, 8 mM MgCl2, 5 mM dithiothreitol, 10% glycerol) at a final volume of 20 μL. The reaction mixtures were incubated at 30 C for 2 h and heated to 90 C for 3 min, and then 5 μL of the stop buffer (90% formamide, 20 mM EDTA, 0.02% bromophenol blue) was added to each reaction mixture. The RNA products were analyzed by electrophoresis on a 20% polyacrylamide gel containing 7 M urea in a tris-borate-EDTA buffer, followed by phosphorimaging with a scanner. The quantification of these images was carried out. The images were analyzed. The molecular weight ladders were generated by labeling Tr7, Tr14, Tr21, and Tr25 with [32P] ATP using polynucleotide kinase.

### In Vitro Assay Validation

Based on in silico experiment results, molecules were selected as inhibitor candidates, such as micafungin, verubecestat, ribavirin, and ALS-8112, which were evaluated in an in vitro RNA synthesis assay (FIG. 2). Test molecules were dissolved in 10% dimethyl sulfoxide, and a 10% dimethyl sulfoxide solution was used as a negative control. The 12-nt trailer complementary sequence (TrC12) at the terminal of the antigenome was used as a template in the RNA synthesis assay. Micafungin was identified as preferred candidate among the tested molecules, showing significant inhibition activity toward RSV polymerase, which retained only 29.6% of the activity of the control group.

### Inhibition Comparison of Micafungin, Totrombopag, and Other Inhibitors on Other Viral RNA Polymerases

Micafungin and totrombopag were compared the with other inhibitors against RSV. The results show micafungin is the best inhibitor against RSV among the tested molecules in de novo and primer-based assays. Micafungin was also tested with several other viral RNA polymerases. This includes the polymerases from vesicular stomatitis virus (VSV, from the Rhabdoviridae family), Nipah virus (NiV, from Paramyxoviridae family), variegated squirrel bornavirus (VSBV, from Bornaviridae family), and severe acute respiratory syndrome coronavirus 2 (SARS-COV2, from Coronaviridae). VSV, NiV, and VSBV belong to the order mononegavirales, also known as non-segmented negative-sense (NNS) RNA viruses. At the same time, SARS-COV2 is a positive-sense single-stranded RNA virus. This data indicates that micafungin and totrombopag inhibit both negative-sense and positive-sense RNA viral RNA polymerases from VSV, NiV, VSBV, and SARS-COV2.

Comparison of Micafungin and Totrombopag with Other Inhibitors

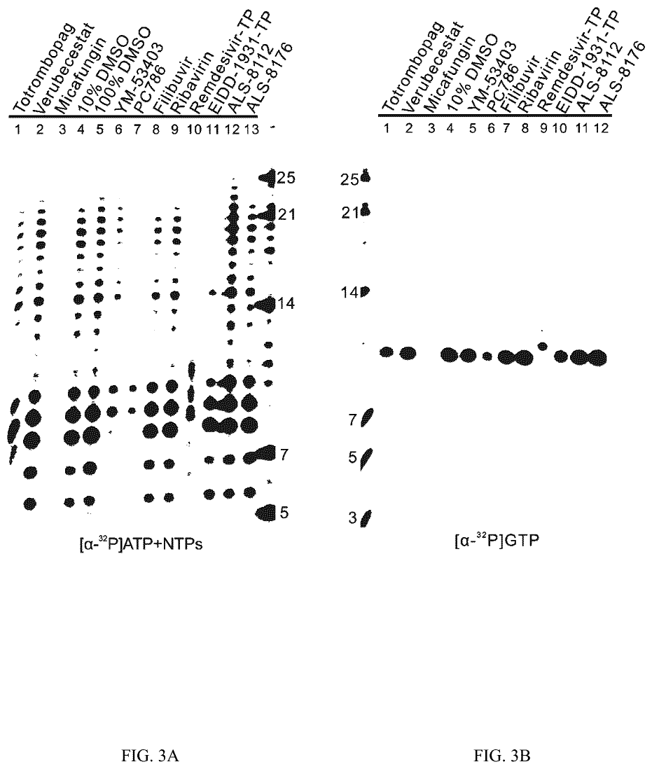

The de novo RNA synthesis activities of RSV polymerase (L-P) towards the template TrC12 were tested adding different molecules, including totrombopag, verubecestat, micafungin, YM-53403, PC786, filibuvir, ribavirin, remdesivir-TP, EIDD-1931-TP, ALS-8112, and ALS-8176 (FIG. 3A). The results show that the de novo RNA synthesis activity of RSV polymerase in the presence of micafungin is the lowest, indicating micafungin is the best inhibitor towards the de novo RNA synthesis activity of RSV polymerase among the tested molecules. Primer-based RNA synthesis activities of RSV polymerase (L-P) with the template TrC12 and primer Tr4 (5′-ACGA) was tested in the presence of radioactively labeled [α-32P] ATP adding the same molecules (FIG. 3B). The results also indicate that micafungin is also the best inhibitors towards the primer-based RNA synthesis activity of RSV polymerase among the tested molecules.

The time course testing of 0, 25, and 50 μM Micafungin using TrC12 as a template indicates that the impact of Micafungin on the RSV polymerase activity is time and concentration dependent. The RNA products are increasing along with the time and the activity of RSV polymerase in the presence of 50 μM Micafungin is the lowest at all time points.

Testing with Viral RNA Polymerases from Different Viruses

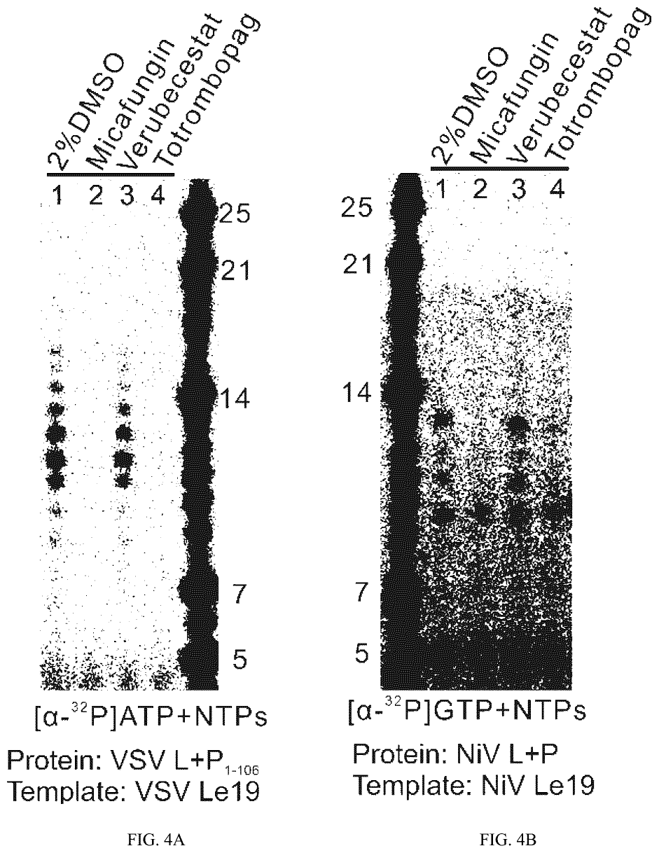

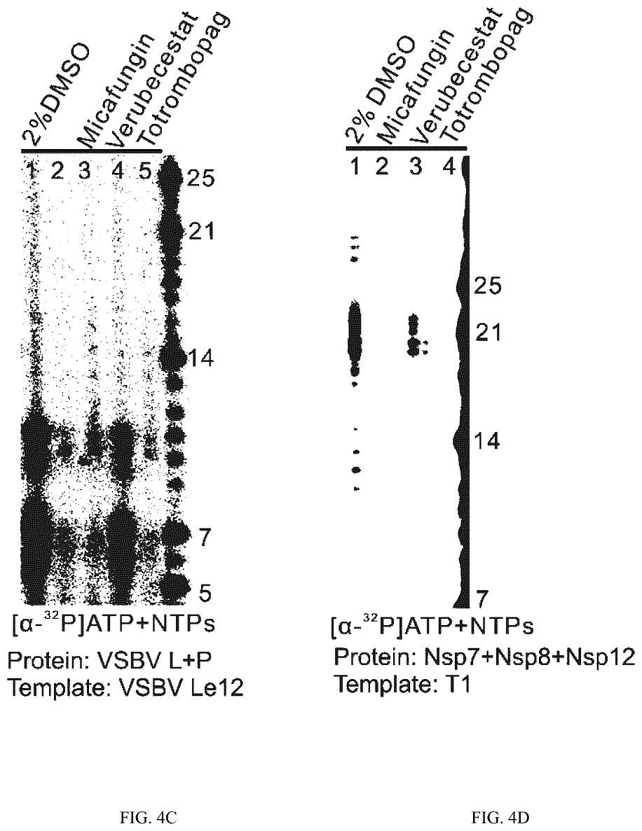

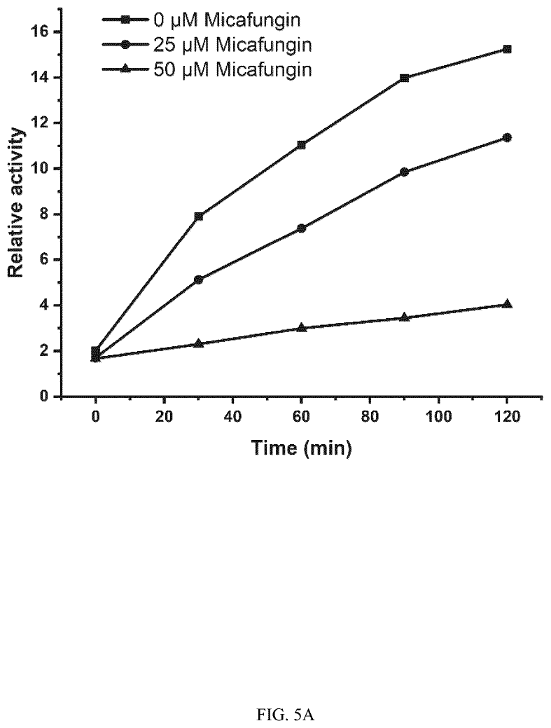

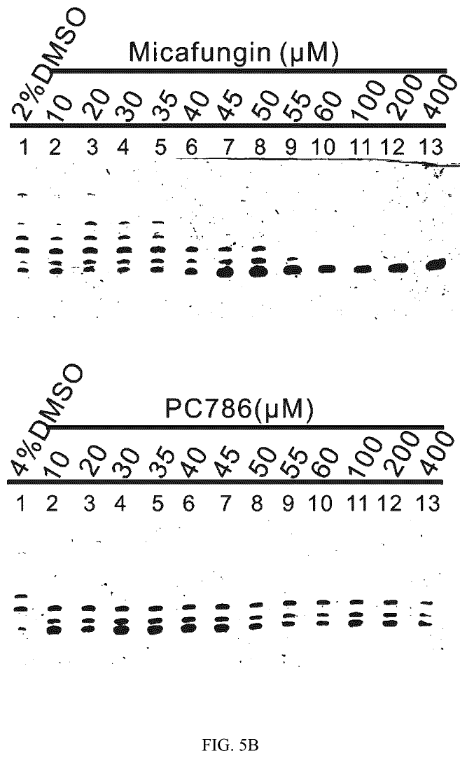

To investigate the effect of Micafungin on viral RNA polymerases from other viruses, the RNA polymerases of VSV, NiV, VSBV, and SARS-COV2 we purified. Exposure of the compounds to RNA polymerases were tested in vitro RNA synthesis assays. The results indicate that micafungin and totrombopag both inhibit the RNA synthesis activities of the RNA polymerases from VSV, NiV, VSBV, and SARS-COV2, and that micafungin is superior (FIG. 4).

Micafungin shows the most inhibition towards RSV polymerase in both de novo and primer-based RNA synthesis activities compared to totrombopag, verubecestat, YM-53403, PC786, filibuvir, ribavirin, remdesivir-TP, EIDD-1931-TP, ALS-8112, and ALS-8176. In addition, micafungin and totrombopag both inhibit the RNA synthesis activities of the RNA polymerases from VSV, NiV, VSBV, and SARS-COV2. These viral RNA polymerases are from different viruses, including positive-sense and negative-sense RNA viruses, indicating they are broad-spectrum antiviral molecules against the polymerase of RNA viruses.

1. A method of treating or preventing a viral infection comprising administering an effective amount an RNA-dependent RNA Polymerase (RdRP) inhibitory compound, derivative, or salt thereof to a subject in need thereof, wherein the compound is selected from: micafungin totrombopag, or verubecestat.

2. The method of claim 1, wherein the compound is micafungin.

3. The method of claim 1, wherein the compound is totrombopag.

4. The method of claim 1, wherein the compound is verubecestat.

5. The method of claim 1, wherein the viral infection is a positive-sense virus or negative-sense RNA virus.

6. The method of claim 5, wherein the virus is respiratory syncytial virus (RSV), vesicular stomatitis virus (VSV), Nipah virus (NiV), variegated squirrel bornavirus (VSBV), coronavirus, severe acute respiratory syndrome coronavirus 2 (SARS-COV2), rabies virus (RABV), or Ebola virus (EBOV).

7. The method of claim 1, wherein the compound is administered in combination with an anti-viral agent or vaccine.

8. A method of treating or preventing a respiratory syncytial virus (RSV) infection comprising administering an effective amount micafungin or salt thereof to a subject in need thereof.

9. The method of claim 7, wherein micafungin is administered in combination with an anti-RSV agent.

10. The method of claim 9, wherein the anti-RSV agent is ribavirin, lumicitabine, YM-53403, or a recombinant RSV antibody.

11. The method of claim 10, wherein the recombinant RSV antibody is palivizumab or nirsevimab.

12. The method of claim 9, wherein the subject is diagnosed with RSV and a newborn less than 1 years old.

13. A method of treating or preventing a severe acute respiratory syndrome coronavirus 2 (SARS-COV2) or other coronavirus infection comprising administering an effective amount of micafungin or salt thereof to a subject in need thereof.

14. A method of treating or preventing a vesicular stomatitis virus (VSV) infection comprising administering an effective amount of micafungin or salt thereof to a subject in need thereof.

15. A method of treating or preventing a nipah virus (NiV) infection comprising administering an effective amount of micafungin or salt thereof to a subject in need thereof.

16. A method of treating or preventing an ebola virus (EBOV) infection comprising administering an effective amount of micafungin or salt thereof to a subject in need thereof.

17. A method of treating or preventing a variegated squirrel bornavirus (VSBV) infection comprising administering an effective amount of micafungin or salt thereof to a subject in need thereof.

18. A method of treating or preventing a rabies virus (RABV) infection comprising administering an effective amount of micafungin or salt thereof to a subject in need thereof.

19. A pharmaceutical composition for use in treating a viral infection comprising micafungin or salt thereof.

20. The pharmaceutical composition of claim 19, further comprising another anti-viral agent.
